(19) United States
(12) Patent Application Publication
(10) Pub. No.: US 2023/0142643 A1
(43) Pub. Date: May 11, 2023

(54) RADIATION IMAGING APPARATUS

(71) Applicant: CANON KABUSHIKI KAISHA, Tokyo (JP)

(72) Inventors: Kaito Uehata, Kanagawa (JP); Motoki Tagawa, Kanagawa (JP)

(21) Appl. No.: 18/053,306

(22) Filed: Nov. 7, 2022

(30) Foreign Application Priority Data
Nov. 11, 2021 (JP) 2021-184236

(51) Int. Cl. G01T 1/24; G01T 1/02

(52) U.S. Cl. CPC G01T 1/247; G01T 1/026; G01T 1/245

(57) ABSTRACT

A radiation imaging apparatus comprises an imaging unit including an effective region including pixels for generating a radiation image based on irradiated radiation and a receptor field region including pixels for measuring a dose of the radiation, and a control unit configured to output a signal for controlling irradiation of the radiation by comparing the measured dose with a threshold. An effective region index representing the effective region and a receptor field index representing the receptor field region are identifiably formed on a radiation incident surface of the imaging unit.

### BACKGROUND

### Field of the Disclosure

The present disclosure relates to a radiation imaging apparatus.

### Description of Related Art

In recent years, as a radiation imaging apparatus used for medical imaging diagnosis, an apparatus that obtains a digital image using a semiconductor sensor is becoming widespread. In the field of the radiation imaging apparatus, a technique called AEC (Auto Exposure Control) (to be referred to as an AEC function hereinafter) is used for obtaining an image of a proper optical density, and for managing a radiation dose at the time of imaging. The AEC function is a function of monitoring the dose of irradiated radiation, and controlling irradiation of the radiation when the dose reaches a defined threshold.

Japanese Patent Laid-Open No. 2018-50828 discloses a technique in which the user can readily align an object with an imaging unit by providing a pixel region for dose management and a pixel region for image generation in the imaging unit, and displaying the pixel region for dose management on the imaging unit.

In the technique described in Japanese Patent Laid-Open No. 2018-50828, a receptor field can be changed in accordance with an imaging portion of the object, and thus it may be necessary to display a plurality of receptor fields when directly displaying the receptor fields on the main body of the imaging unit. In addition, an effective region and an index indicating the center of the effective region are displayed together.

To capture a radiation image, the user first aligns the object in accordance with the display of the receptor field. After that, the imaging unit and the object are irradiated with guide light that illuminates an irradiation range with visible light, thereby adjusting the radiation irradiation range by comparing the end portions and center lines of the guide light with the display of the effective region. In the alignment operation, the user visually confirms the display of the effective region from the position of the radiation imaging apparatus. Therefore, if the direction and position of the radiation imaging apparatus are changed, it may be difficult to discriminate between the display of the effective region and the display of the receptor field.

### SUMMARY OF THE DISCLOSURE

The present disclosure provides a radiation imaging apparatus to which an effective region index representing an effective region and a receptor field index representing a receptor field region are identifiably formed on a radiation incident surface of the imaging unit.

According to one aspect of the present disclosure, there is provided a radiation imaging apparatus comprising an imaging unit including an effective region including pixels for generating a radiation image based on irradiated radiation and a receptor field region including pixels for measuring a dose of the radiation, and a control unit configured to output a signal for controlling irradiation of the radiation by comparing the measured dose with a threshold, wherein an effective region index representing the effective region and a receptor field index representing the receptor field region are identifiably formed on a radiation incident surface of the imaging unit.

### BRIEF DESCRIPTION OF THE DRAWINGS

FIG. 1 is a block diagram showing the arrangement of a radiation imaging apparatus according to the first embodiment;

FIG. 2 is a flowchart for explaining the procedure of the processing of the radiation imaging apparatus according to the first embodiment;

FIG. 3 shows views for explaining adjustment of a radiation irradiation range at the time of imaging;

FIG. 4 shows views of examples of region indices of the radiation imaging apparatus according to the first embodiment;

FIG. 5 is a view showing examples of region indices of the radiation imaging apparatus according to the first embodiment;

FIG. 6 is a view showing examples of region indices of the radiation imaging apparatus according to the first embodiment; and

FIG. 7 shows views showing examples of region indices formed on the surface of a region index sheet member according to the second embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to any specific embodiment that requires all such features, and multiple such features may be combined as appropriate. Furthermore, in the attached drawings, the same reference numerals are given to the same or similar configurations, and redundant description thereof is omitted.

Details of sizes and structures shown in the respective embodiments are not limited to the specification and drawings. Note that in this specification, radiation includes not only X-rays but also α-rays, β-rays, γ-rays, particle rays, and cosmic rays.

### First Embodiment

### Example of Arrangement of Radiation Imaging Apparatus

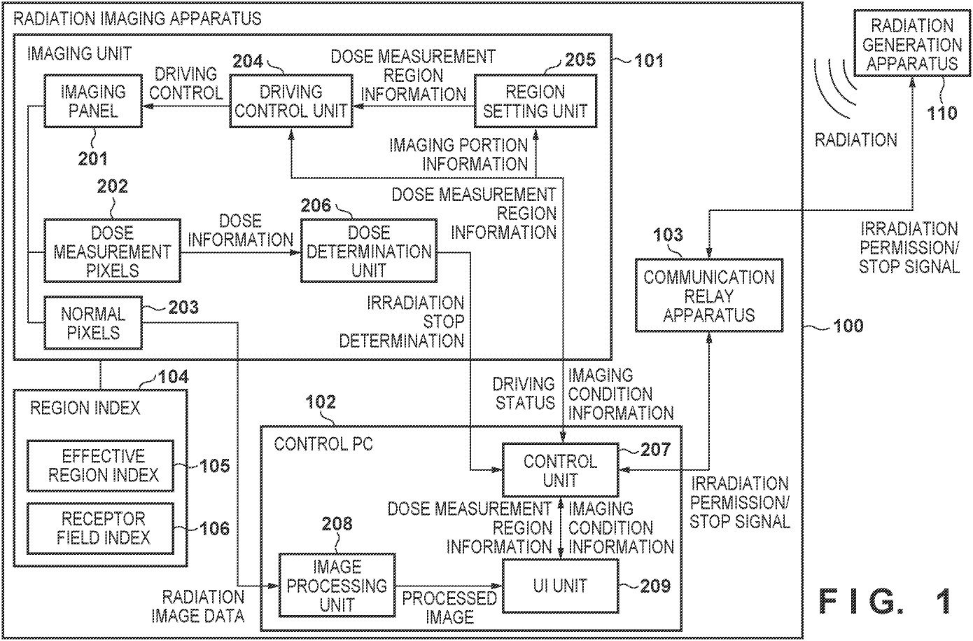

The first embodiment will describe an example of the arrangement of a radiation imaging apparatus that performs imaging using an imaging unit on which a receptor field corresponding to an imaging portion is displayed. FIG. 1 is a block diagram showing the arrangement of a radiation imaging apparatus according to the first embodiment. As shown in FIG. 1, a radiation imaging apparatus 100 includes an imaging unit 101 in operative communication with a control PC 102 (control apparatus), and a communication relay apparatus 103. A radiation generation apparatus 110 is provided as a radiation source separately from the radiation imaging apparatus 100. A region index 104 including an effective region index 105 and a receptor field index 106 is provided on a radiation incident surface of the imaging unit 101.

The effective region index 105 is an index representing a pixel region (effective region) including pixels for generating a radiation image based on radiation with which the radiation incident surface of the imaging unit 101 is irradiated. The effective region index 105 is an index representing an imaging range. The receptor field index 106 is an index representing a pixel region including pixels for measuring the radiation dose and a dose measurement region (receptor field region) corresponding to the imaging portion (imaging portion information). The effective region index 105 representing the effective region and the receptor field index 106 representing the receptor field region are identifiably formed (drawn) on the radiation incident surface of the imaging unit 101. In this embodiment, the effective region index 105 and the receptor field index 106 are identifiably given (drawn) to the radiation incident surface by a method such as a visually confirmable printing method.

The imaging unit 101 is irradiated from the radiation generation apparatus 110, and then generates radiation image information based on a radiation signal having passed through an object. The imaging unit 101 further has a function of monitoring the dose of the radiation emitted from the radiation generation apparatus 110.

The control PC 102 can receive imaging condition information from the user via a user interface (the UI unit 209), and drive the imaging unit 101 based on the imaging condition information. Furthermore, the control PC 102 can output, to a monitor or the like, the radiation image information received from the imaging unit 101. The control PC 102 can control, via the communication relay apparatus 103, the imaging timing of the imaging unit 101 and the radiation irradiation timing of the radiation generation apparatus 110. The communication relay apparatus 103 relays communication between the radiation generation apparatus 110 and the control PC 102.

The user can input an imaging condition via the UI unit 209 of the control PC 102, and also instruct radiation irradiation using an operation board provided in the radiation generation apparatus 110. The radiation generation apparatus 110 irradiates the object and the imaging unit 101 with radiation at a timing of receiving an irradiation permission signal from the control PC 102 via the communication relay apparatus 103.

The control PC 102 can control radiation irradiation by comparing the measured dose with a threshold, and the radiation generation apparatus 110 stops radiation irradiation at a timing of receiving an irradiation stop signal from the control PC 102 via the communication relay apparatus 103.

The imaging unit 101 includes an imaging panel 201, a driving control unit 204 for controlling driving of the imaging panel 201, a region setting unit 205 for generating dose measurement region information and outputting it to the driving control unit 204, and a dose determination unit 206 for performing irradiation stop determination based on dose information from a dose measurement pixel 202.

On the imaging panel 201, pixels each including an image sensor for outputting a radiation signal corresponding to the irradiated radiation (incident light) are arranged in an array (a region of a two-dimensional plane). The photoelectric conversion element of each pixel converts, into a radiation signal (charges) as an electrical signal, light converted by a phosphor, and the capacitor of each pixel accumulates the radiation signal (charges). For example, as the phosphor of the imaging panel 201, CsI:Tl obtained by adding thallium (Tl) to cesium iodide (CsI), a terbium-activated rare earth sulfide-based phosphor (for example, G2O2S:Tb), or the like can be used. The imaging panel 201 includes pixels (normal pixels 203) for generating a radiation image based on radiation transmitted through the object and pixels (the dose measurement pixels 202) for measuring the dose of the radiation. The normal pixel 203 accumulates a radiation signal transmitted through the object, thereby generating a radiation image. The dose measurement pixel 202 periodically reads out a radiation signal, and monitors the irradiation dose. The output unit of the imaging panel 201 reads out a signal from each pixel and outputs it under the control of the driving control unit 204.

The normal pixel 203 outputs the radiation signal (radiation image information) to the image processing unit 208 of the control PC 102. The dose measurement pixel 202 outputs the radiation signal (dose information) to the dose determination unit 206. The normal pixel 203 and the dose measurement pixel 202 have different signal lines and gate lines, and the driving control unit 204 can drive the dose measurement pixel 202 and the normal pixel 203 at different timings. If the driving control unit 204 supplies a driving signal to each pixel, a radiation signal (charges) converted by the photoelectric conversion element of each pixel is accumulated. In accordance with a signal from the driving control unit 204, information (radiation image information or dose information) based on the accumulated radiation signal (charges) is output from each pixel at a different timing.

With respect to the pixel arrangement on the imaging panel 201, a form in which the imaging panel 201 formed by the plurality of normal pixels 203 and the imaging panel 201 formed by the plurality of dose measurement pixels 202 are arranged to have a layer structure or a form in which the dose measurement pixels 202 and the normal pixels 203 are mixed and arranged on the two-dimensional plane of the imaging panel 201 may be adopted. Furthermore, the normal pixel 203 and the dose measurement pixel 202 may be arranged as different pixels or one pixel may include an area functioning as the normal pixel 203 and an area functioning as the dose measurement pixel 202.

The driving control unit 204 outputs, to the imaging panel 201, the driving signal generated based on the imaging condition information received from the control unit 207 in the control PC 102, thereby driving the imaging panel 201. The imaging condition information includes, for example, imaging portion information (chest, abdominal part, lumbar spine, or the like), an imaging direction (PA (posterior-anterior)/AP (anterior-posterior) or front/side), and object information (a physical constitution, information indicating whether the object is an infant, or the like).

If the imaging condition information is input from a control unit 207 to the driving control unit 204, the driving control unit 204 generates a driving control signal for driving each pixel (the dose measurement pixel 202 or the normal pixel 203) of the imaging panel 201. The driving control unit 204 controls driving of the normal pixel 203 to accumulate radiation signals from the start to the end of radiation irradiation and output the accumulated radiation signals.

In accordance with the imaging portion information of the object, the region setting unit 205 sets a pixel region (dose measurement region) to be used to measure the dose. That is, the region setting unit 205 receives the imaging condition information from the control unit 207, selects, based on the received information, dose measurement region information preset for each imaging portion, and outputs the selected information to the driving control unit 204 and the control unit 207. The control unit 207 outputs, to a UI unit 209, the dose measurement region information obtained from the region setting unit 205.

The driving control unit 204 specifies, based on the dose measurement region information obtained from the region setting unit 205, the pixel region of the dose measurement pixels 202 to be used for dose measurement from the dose measurement pixels arranged on the imaging panel 201, and instructs, based on the driving control signal for the dose measurement pixel, to execute dose monitoring driving of periodically performing a readout operation for the specified pixel region of the dose measurement pixels 202. Then, the driving control unit 204 controls driving of the dose measurement pixels 202 in the dose measurement region so as to periodically read out the radiation signals and output them to the dose determination unit 206.

The dose determination unit 206 receives the integration value of the doses from the dose measurement pixels 202, compares the integration value with a preset dose threshold, and outputs, if the integration value exceeds the dose threshold, a radiation irradiation stop determination signal to the control unit 207 of the control PC 102. If the integration value exceeds the threshold as a result of comparison of the integration value of the doses measured by the pixels in the pixel region with the preset threshold, the control PC 102 controls to stop radiation irradiation.

The control PC 102 includes the control unit 207, an image processing unit 208 for performing image processing for a radiation image, and the user interface (UI) unit 209. The control unit 207 outputs the imaging condition information for the imaging unit 101 to the driving control unit 204 based on the imaging condition information input from the user via the UI unit 209.

The control unit 207 receives a driving status output from the driving control unit 204. After the imaging panel 201 is powered on, the driving control unit 204 checks the output characteristic of the imaging panel 201, and performs preparation driving of the imaging panel 201 until the output characteristic of the imaging panel 201 becomes stable. The driving control unit 204 transmits a driving status indicating an imaging disable state to the control unit 207 until the output characteristic of the imaging panel 201 becomes stable. After the output characteristic of the imaging panel 201 becomes stable, the driving control unit 204 transmits a driving status indicating an imaging enable state to the control unit 207. Furthermore, the control unit 207 communicates with the radiation generation apparatus 110 via the communication relay apparatus 103 to transmit an irradiation permission/stop signal.

The image processing unit 208 applies, to the radiation image information received from the normal pixel 203 in the imaging unit 101, image processing such as offset correction processing, sensitivity correction processing, spatial frequency processing, gradation processing, and defective pixel correction processing.

The UI unit 209 can receive the processed radiation image output from the image processing unit 208 and output the received image to a display device such as a monitor while transferring the image to the server of a in-hospital image management system such as PACS (Picture Archiving and Communication Systems). Furthermore, the UI unit 209 can output, to the control unit 207, the imaging condition information input from the user. The control unit 207 obtains the dose measurement region information output from the region setting unit 205, and outputs it to the UI unit 209. The UI unit 209 can receive the dose measurement region information from the control unit 207, and display a dose measurement region based on the imaging portion on an output device such as a monitor. For example, the UI unit 209 can function as a display control unit for controlling display of a display unit such as a monitor, and output, to an output device such as a monitor, an image obtained by superimposing the dose measurement region information on the image output from the image processing unit 208 after the image processing. That is, the UI unit 209 (display control unit) can display, on the display unit, the image in a display form in which the dose measurement region set by the region setting unit 205 is superimposed on the image having undergone the image processing. By performing such output control (display control), the user can determine, based on the output (displayed) image, whether the dose measurement region has appropriately been set for the imaging portion. It is also possible to compare the image with an image captured in the past under the same imaging condition.

### Procedure of Processing of Radiation Imaging Apparatus 100

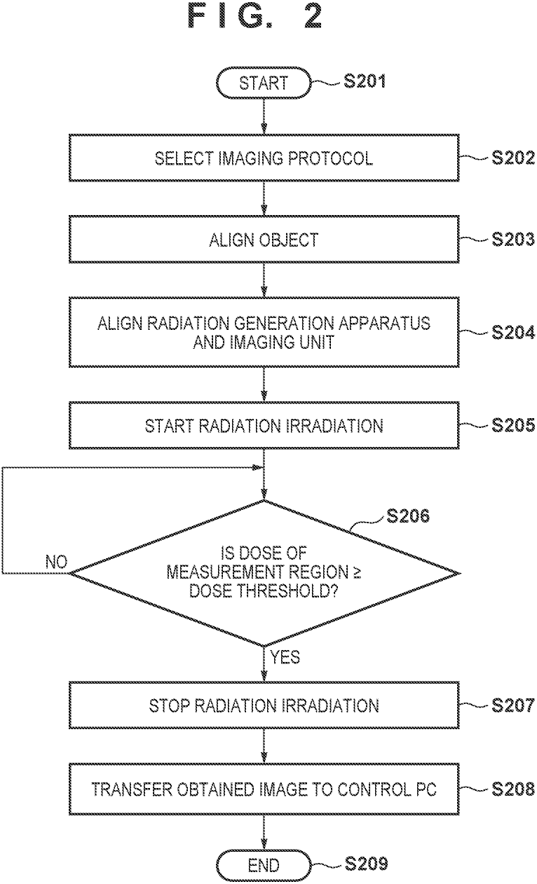

The procedure of the processing of the radiation imaging apparatus 100 according to the first embodiment will be described next with reference to FIG. 2. In step S201, the imaging unit 101 is powered on. At this time, the imaging panel 201 is also powered on. At this time, the power of the control PC 102 needs to be ON. The driving control unit 204 starts preparation driving for starting imaging under the control of the control unit 207. In this preparation driving, the imaging unit 101, the control PC 102, and the communication relay apparatus 103 are controlled so that radiation imaging cannot be started before the output characteristic of the imaging panel 201 becomes stable.

In step S202, the user selects an imaging protocol via the UI unit 209. The imaging protocol is an imaging application installed in the control PC 102, and is an imaging application that is prepared in advance for each imaging condition and dedicated for the radiation imaging apparatus 100. The imaging protocol includes imaging condition information such as an imaging portion, an imaging direction (PA (posterior-anterior)/AP (anterior-posterior) or front/side), and object information (a physical constitution or information indicating whether the object is an infant).

Next, the imaging condition information is output from the UI unit 209 to the driving control unit 204 and the region setting unit 205 via the control unit 207. If the imaging condition information including imaging portion information is input from the control unit 207 to the region setting unit 205, the region setting unit 205 selects dose measurement region information stored in advance and corresponding to the imaging portion information, and outputs it to the driving control unit 204.

In step S203, the user aligns the imaging portion of the object with the imaging unit 101. The user aligns the imaging portion of the object with the receptor field index 106 corresponding to the imaging condition information selected in step S202.

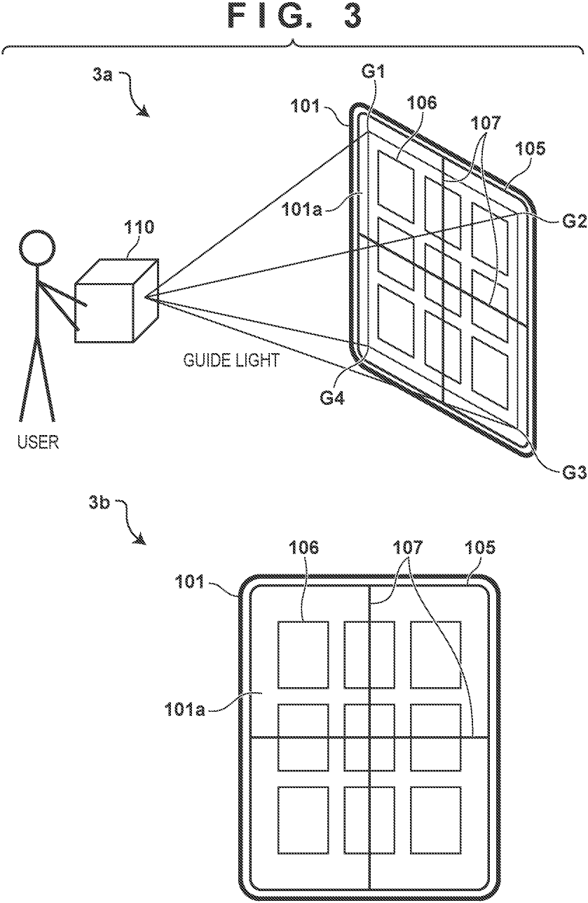

FIG. 3 shows views for explaining adjustment of a radiation irradiation range at the time of imaging according to the first embodiment. In FIG. 3, 3a is a view for schematically explaining an example of adjustment of the radiation irradiation range, in which the object is not illustrated. In FIG. 3, 3b is a view exemplifying the effective region index 105 and the receptor field indices 106 formed on a radiation incident surface 101a of the imaging unit 101. In the example shown in 3b of FIG. 3, the receptor field indices 106 are formed in an arrangement of 3 rows × 3 columns but are not limited to this example. Various receptor field indices 106 corresponding to the imaging portions (pieces of imaging portion information) may be formed in other arrangements. As alignment indices, center lines 107 indicating the center positions of the radiation incident surface 101a in the longitudinal direction and the widthwise direction are formed on the radiation incident surface 101a of the imaging unit 101.

In step S204, the user aligns the radiation irradiation range by the radiation generation apparatus 110 with the effective region index 105 in the imaging unit 101, as shown in FIG. 3. At this time, guide light as visible light that illuminates the radiation irradiation range is emitted from the radiation generation apparatus 110 (3a of FIG. 3), and the user can adjust the radiation irradiation range of the radiation generation apparatus 110 by aligning the end portions (G1 to G4: 3a of FIG. 3) of the guide light with the effective region index 105.

Since the user adjusts the irradiation range of the guide light by operating the radiation generation apparatus 110, he/she visually confirms the effective region index 105 from a far distance. On the other hand, since the user uses the receptor field index 106 to align the object with the imaging unit 101, he/she visually confirms the receptor field index 106 from a close distance. Therefore, it is required to identifiably give, to the radiation incident surface 101a of the imaging unit 101, the effective region index 105 representing the effective region and the receptor field indices 106 each representing the receptor field region.

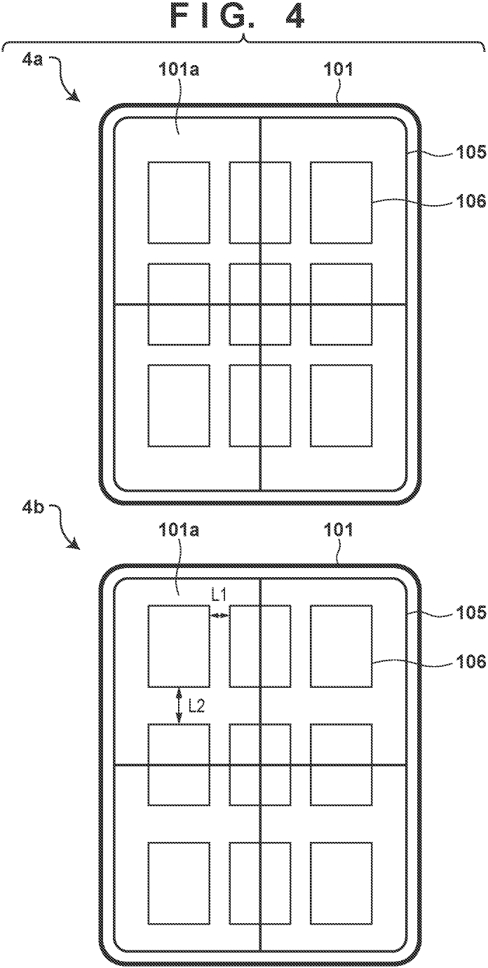

FIGS. 4, 5, and 6 are views each showing examples of region indices (the effective region index 105 and the receptor field indices 106) in the radiation imaging apparatus according to the first embodiment.

As shown in 4a of FIG. 4, for example, the effective region index 105 and the receptor field indices 106 may be formed on the radiation incident surface 101a using lines of different line widths. That is, the width of a line representing the effective region index 105 may be made different from the width of a line representing the receptor field index 106, thereby making it possible to easily identify and distinguish the effective region index 105 and the receptor field index 106.

As a detailed example, the width of the line representing the receptor field index 106 may be set to a first line width (for example, 0.5 mm) or less, and the width of the line representing the effective region index 105 may be set to a second line width (for example, 1.0 mm) or more. The numerical values representing the line widths (the widths of the lines) are merely examples, and the indices may be formed on the radiation incident surface 101a by setting the width of the line representing the effective region index 105 to a value larger than the width of the line representing the receptor field index 106 (second line width > first line width).

In FIG. 4, 4b is a view showing an example in which the receptor field indies 106 having a rectangular shape (for example, an oblong shape) are arrayed in a matrix at different intervals in the vertical and horizontal directions. Reference symbol L1 denotes an arrangement interval of the receptor field indices 106 in the horizontal direction; and L2, an arrangement interval of the receptor field indices 106 in the vertical direction. In 4a of FIG. 4, the arrangement intervals in the vertical and horizontal directions are set almost equal to each other. To the contrary, in 4b of FIG. 4, the arrangement intervals in the vertical and horizontal directions are set different from each other. By setting the arrangement intervals L1 and L2 different from each other, as shown in 4b of FIG. 4, it is possible to facilitate selection of the optimum receptor field index 106 in accordance with, for example, the size of the object and the imaging portion while identifying the effective region index 105 and the receptor field index 106 by the difference in line width.

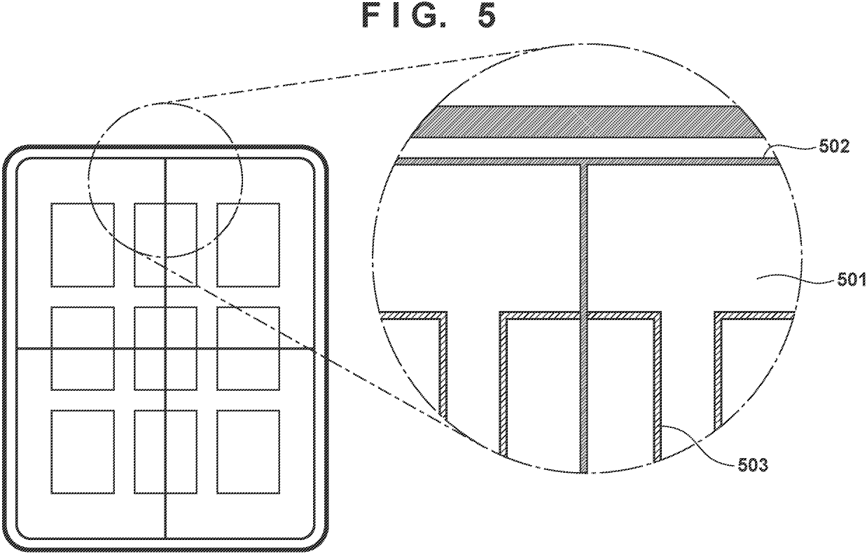

As shown in FIG. 5, the color scheme (first color scheme) of the radiation incident surface 101a of the imaging unit 101, the color scheme (second color scheme) of the line representing the effective region index 105, and the color scheme (third color scheme) of the line representing the receptor field index 106 may be given by a combination of different color schemes. Especially, in consideration of the visibility of the guide light, a first color scheme 501 of the radiation incident surface 101a may be white, a second color scheme 502 of the line representing the effective region index 105 may be black, and a third color scheme 503 of the line representing the receptor field index 106 may be gray.

Note that the combination of colors is merely an example, and can be changed so as to readily identify the effective region index 105, as compared with the receptor field index 106. Any color scheme can be used for each of the effective region index 105 and the receptor field index 106, as long as both are readily identifiable and uniquely distinguishable from each other.

The effective region index 105 and the receptor field index 106 may be formed on the radiation incident surface 101a using lines of different hues. That is, the hue of the line representing each region is made different, thereby making it possible to identify the effective region index 105 and the receptor field index 106.

Note that the feature of the color is not limited to the hue and may be brightness or chroma. The brightness or chroma of the line representing each region can be made different to identify the effective region index 105 and the receptor field index 106, and it is also possible to change the combination of colors so as to readily identify the effective region index 105, as compared with the receptor field index 106. That is, the effective region index 105 and the receptor field index 106 may be formed on the radiation incident surface 101a using lines of different brightnesses or different chromas. Lines between which at least one of the hue, brightness, and chroma as a combination of the features of color is different may be used to form the indices on the radiation incident surface 101a.

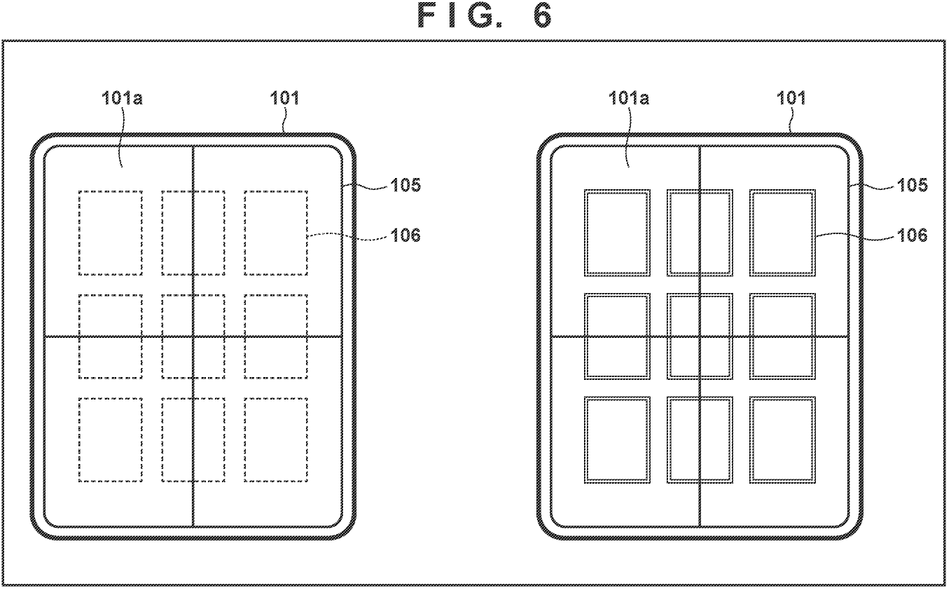

As shown in FIG. 6, the effective region index 105 and the receptor field index 106 may be formed on the radiation incident surface 101a using different line types. That is, the line types of the effective region index 105 and the receptor field index 106 may be made different from each other, thereby making it possible to identify the effective region index 105 and the receptor field index 106. The line types include a solid line, a broken line, a double line, a one-dot dashed line, and a two-dot dashed line. For example, as shown in FIG. 6, the effective region index 105 may be given using a solid line, and the receptor field index 106 may be given using a broken line or a double line.

Referring back to FIG. 2, in step S205, after inputting an irradiation condition using the operation board of the radiation generation apparatus 110, the user presses an irradiation button to start to irradiate the object and the imaging unit 101 with radiation. The control unit 207 obtains the driving status from the driving control unit 204 of the imaging unit 101, and confirms that the driving status indicates an imaging enable state. Upon receiving the driving status indicating an imaging enable state from the driving control unit 204, the control unit 207 instructs the driving control unit 204 to start driving of accumulating a radiation signal, and permits radiation irradiation by sending a radiation irradiation permission signal to the radiation generation apparatus 110 via the communication relay apparatus 103.

After the imaging condition information is input from the control unit 207 to the driving control unit 204, the driving control unit 204 generates a driving control signal for driving each pixel (the dose measurement pixel 202 or the normal pixel 203) of the imaging panel 201. The driving control unit 204 instructs the normal pixel 203 to accumulate a radiation signal based on the driving control signal for the normal pixel.

Furthermore, the driving control unit 204 specifies a pixel region of pixels to be used for dose measurement based on information (dose measurement region information) indicating the dose measurement region set by the region setting unit 205, and instructs, based on the driving control signal for the pixels of the specified pixel region, to execute dose monitoring driving of periodically performing a readout operation. That is, the driving control unit 204 specifies, based on the dose measurement region information obtained from the region setting unit 205, a pixel region of the dose measurement pixels 202 to be used for dose measurement among the dose measurement pixels arranged on the imaging panel 201, and instructs, based on the driving control signal for the dose measurement pixel, to execute dose monitoring driving of periodically performing a readout operation for the specified pixel region of the dose measurement pixels 202. Then, with respect to the pixels in the dose measurement region, the driving control unit 204 controls driving of the dose measurement pixels 202 so as to periodically read out the radiation signals and output them to the dose determination unit 206. By controlling driving by the driving control unit 204, the dose measurement pixel 202 periodically outputs dose information to the dose determination unit 206.

In step S206, the dose determination unit 206 accumulates the dose information periodically output from the dose measurement pixel 202, and compares the accumulated dose with the preset dose threshold (threshold) for each imaging portion. If the accumulated dose is smaller than the dose threshold (NO in step S206), the dose determination unit 206 continuously performs the comparison processing in step S206 until the accumulated dose becomes equal to the threshold. On the other hand, if the accumulated dose is equal to or larger than the dose threshold, that is, the accumulated dose matches or exceeds the dose threshold (threshold) (YES in step S206), the dose determination unit 206 outputs radiation irradiation stop determination to the control unit 207.

In step S207, upon obtaining the irradiation stop determination signal from the dose determination unit 206, the control unit 207 outputs a radiation irradiation stop signal to the radiation generation apparatus 110 via the communication relay apparatus 103. Upon receiving the radiation irradiation stop signal from the communication relay apparatus 103, the radiation generation apparatus 110 stops radiation irradiation. Furthermore, the control unit 207 instructs the driving control unit 204 to stop accumulation driving of the normal pixels 203 and stop dose monitoring driving of the dose measurement pixels. After receiving the driving stop instruction from the control unit 207, the driving control unit 204 executes control to stop accumulation driving of the normal pixels 203 and stop dose monitoring driving of the dose measurement pixels.

In step S208, signals from the normal pixels 203 are output, to the image processing unit 208 of the control PC 102, radiation image information based on the accumulated radiation signals (charges). The image processing unit 208 performs, for the obtained radiation image information, image processing such as offset correction processing, sensitivity correction processing, spatial frequency processing, gradation processing, and defective pixel correction processing, and outputs, to the UI unit 209, the image (processed image) having undergone the image processing. The control unit 207 obtains the dose measurement region information from the region setting unit 205, and outputs it to the UI unit 209. The UI unit 209 can function as a display control unit, and output (display), to the output device such as a monitor, an image in a display form in which the dose measurement region information obtained from the control unit 207 is superimposed on the image (processed image) having undergone the image processing. This allows the user to confirm the result of aligning the imaging portion of the object with the dose measurement region. After the control unit 207 outputs the dose measurement region information and the processed image to the output device, the process advances to step S209, thereby terminating imaging.

In the radiation imaging apparatus 100 according to this embodiment, the effective region index 105 representing the effective region including pixels for generating a radiation image based on radiation with which the radiation incident surface of the imaging unit 101 is irradiated and the receptor field index 106 representing the receptor field region including pixels for measuring the dose of radiation are identifiably formed on the radiation incident surface. This allows the user to identify the effective region index 105 and the receptor field index 106, and the user can align the irradiation range of the guide light with the effective region index 105 by operating the radiation generation apparatus 110 while confirming the effective region index 105. In addition, the user can align the object with the imaging unit 101 by confirming the receptor field index 106.

### Second Embodiment

The second embodiment will describe a case in which a region index 104 (an effective region index 105 and a receptor field index 106) is formed on a region index sheet member 114, and the region index sheet member 114 is attached to a radiation incident surface 101a of an imaging unit 101 and used. The region index sheet member 114 can be attached to the radiation incident surface 101a of the imaging unit 101, the effective region index 105 is drawn on the region index sheet member 114, and the receptor field index 106 is formed on the region index sheet member 114 by an unevenness formed to be identifiable at least visually or tactilely.

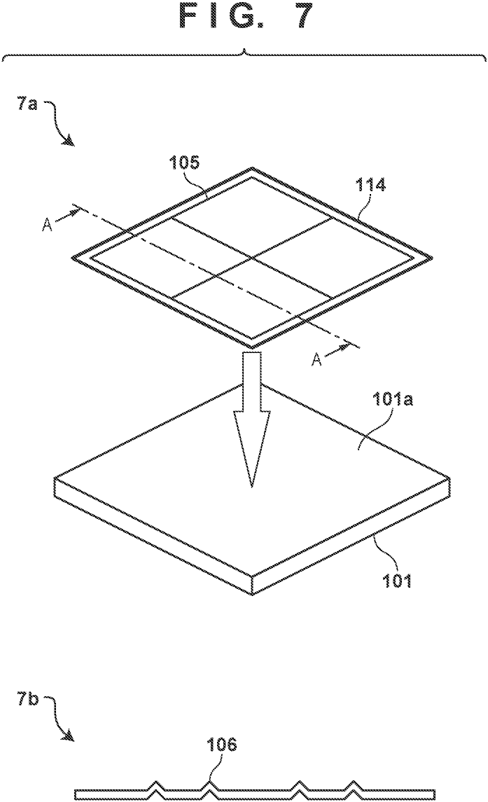

In FIG. 7, 7a is a view showing examples of the region indices (the effective region index 105 and the receptor field index 106) formed on the surface of the region index sheet member 114 according to the second embodiment, and 7b is a view exemplifying the arrangement of the receptor field index 106 taken along a line A - A in 7a.

The region index sheet member 114 can transmit radiation emitted from the radiation generation apparatus 110, and is pasted and attached to the radiation incident surface 101a of the imaging unit 101. A procedure of imaging a radiation image is the same as in the first embodiment and a description thereof will be omitted.

In this embodiment, the effective region index 105 is drawn on the surface of the region index sheet member 114 by a method such as a visually confirmable printing method. A method of forming the effective region index 105 on the radiation incident surface 101a is by drawing a line, as described in the first embodiment. In drawing a line, for example, the line type or the features (hue, brightness, and chroma) of color may be changed in accordance with imaging portion information, thereby forming (drawing) the effective region index 105 on the surface of the region index sheet member 114. The user can align the irradiation range of guide light with the effective region index 105 by operating the radiation generation apparatus 110 while confirming the effective region index 105 of the region index sheet member 114.

Furthermore, as shown in 7b of FIG. 7, the receptor field index 106 has a structure in which a raised unevenness similar to braille dots is formed on the surface of the region index sheet member 114, and the raised unevenness is configured identify the receptor field index 106 at least visually or tactilely. The unevenness given as the receptor field index 106 is not limited to the arrangement in which unevenness is formed at a predetermined interval, as shown in 7b of FIG. 7, and may be given by forming such unevenness that the vertices of convex portions are arranged in a line. The receptor field index 106 may be formed on the surface of the region index sheet member 114 by convex portions (e.g., ridges) or by concave portions (grooves). For example, at least one (to be referred to as an unevenness pattern) of the unevenness, the convex portion, and the concave portion may be changed in accordance with imaging portion information, thereby forming the receptor field index 106 on the surface of the region index sheet member 114.

When the region index sheet member 114 having undergone unevenness processing, more specifically, a resin sheet having unevenness, as shown in 7b of FIG. 7, is attached (pasted, glued or fused) to the radiation incident surface 101a of the imaging unit 101, the receptor field index 106 is formed on the radiation incident surface 101a by the unevenness pattern formed to be identifiable at least visually or tactilely. Thus, the effective region index 105 and the receptor field index 106 representing the receptor field region including pixels for measuring the dose of radiation are identifiably formed on the radiation incident surface 101a. When performing alignment of the object, the user can align the object with the imaging unit 101 by confirming the receptor field index 106.

Note that in the arrangement of the region index sheet member 114 according to this embodiment, unevenness may be formed on the housing of the imaging unit 101 so as to readily identify the receptor field index 106. That is, the effective region index 105 may be formed on the radiation incident surface 101a of the imaging unit 101 by a method (for example, drawing) such as a visually confirmable printing method, and the receptor field index 106 may be formed on the radiation incident surface 101a of the imaging unit 101 using the unevenness pattern, such as ridges formed to be identifiable at least visually or tactilely.

A grid may be used to suppress blurring of a radiation image caused by radiation scattering when transmitted through the object. The region index sheet member 114 described in this embodiment may be attached to the surface of the grid. That is, the region index sheet member 114 can be attached to the surface of the grid, the effective region index 105 may be drawn on the region index sheet member 114, and the receptor field index 106 may be formed on the region index sheet member 114 using the unevenness pattern configured to be identifiable at least visually or tactilely.

The arrangement of the region index sheet member 114 may be provided directly on the surface of the grid. For example, the effective region index 105 may be drawn on the surface of the grid attachable to the radiation incident surface 101a of the imaging unit 101, and the receptor field index 106 may be formed on the surface of the grid using the unevenness pattern formed to be identifiable at least visually or tactilely.

According to this embodiment, it is possible to provide a radiation imaging apparatus to which an effective region index representing an effective region and a receptor field index representing a receptor field region are identifiably given. The user can identify the effective region index 105 and the receptor field index 106, and align the irradiation range of guide light with the effective region index 105 by operating the radiation generation apparatus 110 while confirming the effective region index 105. Furthermore, the user can align the object with the imaging unit 101 by confirming the receptor field index 106.

According to the present disclosure, it is possible to provide a radiation imaging apparatus to which an effective region index representing an effective region and a receptor field index representing a receptor field region are identifiably given.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2021-184236, filed Nov. 11, 2021, which is hereby incorporated by reference herein in its entirety.

What is claimed is:

1. A radiation imaging apparatus comprising an imaging unit including an effective region including pixels for generating a radiation image based on irradiated radiation and a receptor field region including pixels for measuring a dose of the radiation, and a control unit configured to output a signal for controlling irradiation of the radiation by comparing the measured dose with a threshold,
wherein an effective region index representing the effective region and a receptor field index representing the receptor field region are formed on a radiation incident surface of the imaging unit.

2. The apparatus according to claim 1, wherein the receptor field index is formed on the radiation incident surface by a line having a line width, and wherein the effective region index is formed on the radiation incident surface by a line having a line width larger than that of the receptor field index.

3. The apparatus according to claim 1, wherein the effective region index and the receptor field index are formed on the radiation incident surface by different line types.

4. The apparatus according to claim 1, wherein the effective region index and the receptor field index are formed on the radiation incident surface by lines of different hues.

5. The apparatus according to claim 4, wherein the effective region index and the receptor field index are formed on the radiation incident surface by lines of different brightnesses.

6. The apparatus according to claim 4, wherein the effective region index and the receptor field index are formed on the radiation incident surface by lines of different chromas.

7. The apparatus according to claim 1, wherein a first color scheme of the radiation incident surface of the imaging unit, a second color scheme of a line representing the effective region index, and a third color scheme of a line representing the receptor field index are given by a combination of different color schemes.

8. The apparatus according to claim 7, wherein the first color scheme of the radiation incident surface is white, the second color scheme of the line representing the effective region index is black, and the third color scheme of the line representing the receptor field index is gray.

9. The apparatus according to claim 1, further comprising a region index sheet member configured to be attachable to at least one of the radiation incident surface of the imaging unit and a surface of a grid,
wherein the effective region index is drawn on the region index sheet member, and
the receptor field region is formed on the region index sheet member by an unevenness pattern configured to be identifiable at least visually or tactilely.

10. The apparatus according to claim 1, wherein
the effective region index is drawn on a surface of a grid attachable to the radiation incident surface of the imaging unit, and
the receptor field region is formed on the surface of the grid by an unevenness pattern configured to be identifiable at least visually or tactilely.

11. The apparatus according to claim 1, wherein
the effective region index is drawn on the radiation incident surface of the imaging unit, and
the receptor field region is formed on the radiation incident surface by an unevenness pattern configured to be identifiable at least visually or tactilely.

12. The apparatus according to claim 1, wherein
the imaging unit includes a phosphor configured to convert incident radiation into visible light, and
a photoelectric conversion element of each pixel converts the visible light into an electrical signal, and accumulates the electrical signal in a capacitor of each pixel.

13. The apparatus according to claim 12, wherein the imaging unit uses, as the phosphor, one of a phosphor obtained by adding thallium to cesium iodide and a terbium-activated rare earth sulfide-based phosphor.
